(19) United States
(12) Patent Application Publication
(10) Pub. No.: US 2021/0259596 A1
(43) Pub. Date: Aug. 26, 2021

(54) SMART OXIMETRY METHOD, SYSTEM THEREOF AND METHOD FOR ASSESSING CARDIOVASCULAR FUNCTION BASED ON BLOOD OXYGEN STATE

(71) Applicant: CHIMEI MEDICAL CENTER, Tainan City (TW)

(72) Inventors: WILLY CHOU, TAINAN CITY (TW); BOR-SHYH LIN, TAINAN CITY (TW)

(21) Appl. No.: 16/798,691

(22) Filed: Feb. 24, 2020

(51) Int. Cl. A61B 5/1455; A61B 5/145; A61B 5/00

(52) U.S. Cl. CPC A61B 5/14553; A61B 5/7264; A61B 5/4884; A61B 5/14546

(57) ABSTRACT

A smart oximetry method, a system thereof and a method for assessing a cardiovascular function based on a blood oxygen state involve acquiring a hemoglobin parameter of a brain tissue of a subject measured using non-intrusive optical detection; calculating monitoring parameters according to the hemoglobin parameter; entering the monitoring parameters into a linear or non-linear prediction system; assessing a blood oxygen state of the subject according to a preset threshold; and assessing a cardiovascular function of the subject according to the blood oxygen state.

### BACKGROUND OF THE INVENTION

### 1. Technical Field

The present invention relates to a smart oximetry method, a system thereof and a method for assessing a cardiovascular function based on a blood oxygen state, and more particularly to a technique about inputting monitoring parameters related to a blood oxygen state of a subject determined according to a hemoglobin parameter of a brain tissue of the subject to a linear or non-linear prediction system that allows informed manual assessment and monitoring of the blood oxygen state of the brain tissue of that subject.

### 2. Description of Related Art

Cardiopulmonary exercise testing, echocardiography and computed tomography angiography are examples of clinically common detection methods for assessment of cardiovascular function. Cardiopulmonary exercise testing is executed under controlled clinical conditions and uses sensors such as a respiratory mask, electrocardiograph electrodes and a sphygmomanometer to measure the cardiovascular function of a subject while the subject is taking certain exercises. For example, Taiwan Utility Model No. M327721, titled “treadmill monitoring blood oxygen concentration and heartbeat” and Taiwan Patent Publication No. 201909843, titled “blood oxygen concentration dynamic portable monitoring device and blood oxygen dynamic management warning system” prior-art devices using this technique.

However, these known methods have some shortcomings, such as those related to bulky equipment, complex sensor configuration, necessitation of experienced medical staff for pre-testing calibration of the gas exchange system, users' limited exercise amount due to breathing problems caused by their use of respiratory masks during testing, and interference from displacement of electrocardiograph electrodes caused by users' sweat. Echocardiography uses ultrasonic detectors to conduct non-invasive assessment of cardiac structure and functions, and estimates blood flow velocity based on the Doppler Effect. However, the practice requires even higher professional discipline from the operating medical for the fact that the detectors tend to be affected by bones and that Doppler imaging for tissue is highly dependent on shooting angulation. Computed tomography angiography is a non-invasive technique that uses electronic beam tomography to identify occlusion in coronary arteries. However, since the concerns raised by significant radiation and contrast media, it is generally not recommended for patients without conditions.

There are also known techniques that check blood oxygen concentration optically, such as Taiwan Patent No. 1637727, titled “systems, devices, and methods for performing trans-abdominal fetal oximetry and/or trans-abdominal fetal pulse oximetry” that uses reflection fluorescence to detect fetuses' blood oxygen saturation.

### SUMMARY OF THE INVENTION

The present invention not only detects a subject's blood oxygen concentration optically but also assesses the subject's blood oxygen state according to preset monitoring parameters.

The present invention provides a smart oximetry method, comprising:

acquiring a hemoglobin parameter related to a brain tissue of a subject, and calculating any one or a combination of the following monitoring parameters according to the hemoglobin parameter: a monitoring parameter I: a time period for the hemoglobin parameter to reach a peak value from an incremental exercise; a monitoring parameter II: an amount of change of the hemoglobin parameter between an initial value and the peak value; a monitoring parameter III: an average rate of change of the hemoglobin parameter from the beginning of the incremental exercise to the peak value; and a monitoring parameter IV: an average rate of change of the hemoglobin parameter from a 60%-point of the entire incremental exercise to the peak value. Then the monitoring parameters are entered into a linear prediction system or a non-linear prediction system for assessing a blood oxygen state of the subject according to a preset threshold.

The present invention further provides a method for assessing a cardiovascular function based on a blood oxygen state, so that when any of the monitoring parameters exceeds the preset threshold, it is determined that the cardiovascular function of the subject is good, and otherwise, it is determined that the cardiovascular function of the subject is not good.

Further, the hemoglobin parameter comprises any one or a combination of a hemoglobin concentration and a tissue oxygen content. Furthermore, the hemoglobin concentration comprises an oxy-hemoglobin concentration, a deoxy-hemoglobin concentration and a total hemoglobin concentration, and the method further comprises beaming a first light wave having a wavelength between 600 nm and 800 nm and a second light wave having a wavelength between 800 nm and 950 nm to the tissue of the subject, receiving a first reflected light wave and a second reflected light wave of the first light wave and the second light wave, finding an oxy-hemoglobin concentration and a deoxy-hemoglobin concentration of the tissue according to the first reflected light wave and the second reflected light wave, and accordingly finding the total hemoglobin concentration and the tissue oxygen content. Preferably, the monitoring parameters are calculated according to the total hemoglobin concentration and the tissue oxygen content.

Further, the tissue of the subject is a brain tissue of the subject.

Further, the linear prediction system or non-linear prediction system uses one of a neural network, a fuzzy theory system and a chaos theory system.

The present invention further provides a smart oximetry device, for detecting a hemoglobin parameter of a tissue in a subject, comprising:

an optical transmitter, for emitting a first light wave having a wavelength between 600 nm and 800 nm and a second light wave having a wavelength between 800 nm and 950 nm to the tissue of the subject, an optical receiver, for receiving a first reflected light wave and a second reflected light wave of the first light wave and the second light wave, and a microprocessor unit, electrically connected to the optical transmitter and the optical receiver, wherein the microprocessor unit controls the optical transmitter to emit the first light wave and the second light wave, and to receive the first reflected light wave and the second reflected light wave, so as to obtain the hemoglobin parameter, the hemoglobin parameter including an oxy-hemoglobin concentration and a deoxy-hemoglobin concentration.

The present invention further provides a smart oximetry system, for detecting a hemoglobin parameter of a tissue in a subject, so as to obtain a blood oxygen state of the subject, comprising the foregoing smart oximetry device and a processor, the processor signally connected to the smart oximetry device.

The processor receives the hemoglobin parameter, and calculates any one or a combination of the following monitoring parameters according to the hemoglobin parameter: a monitoring parameter I: a time period for the hemoglobin parameter to reach a peak value from an incremental exercise; a monitoring parameter II: an amount of change of the hemoglobin parameter between an initial value and the peak value; a monitoring parameter III: an average rate of change of the hemoglobin parameter from the beginning of the incremental exercise to the peak value; and a monitoring parameter IV: an average rate of change of the hemoglobin parameter from a 60%-point of the entire incremental exercise to the peak value. The system also involves entering the monitoring parameters into a linear prediction system or a non-linear prediction system, and assessing a blood oxygen state of the subject according to a preset threshold.

The present invention further provides a program product, for storing an application that is to be installed to execute the smart oximetry method described previously.

With the foregoing technical features, the present invention achieves the following beneficial effects:

1. The present invention optically detects a subject's blood oxygen concentration, and further assesses the subject's blood oxygen state according to the preset monitoring parameters. It then employs a linear or non-linear prediction system such as a neural network, a fuzzy theory system or a chaos theory system according to preset threshold to effectively divide cardiovascular function groups, thereby facilitating medical staff to recommend different exercise training and recovery processes for patients with cardiovascular diseases.

2. The monitoring parameters used in the present invention help the linear or non-linear prediction system to precisely determine the subject's blood oxygen state and has a proven accuracy of up to 90%.

### BRIEF DESCRIPTION OF THE DRAWINGS

FIG. 1 is a schematic drawing of a smart oximetry system of the present invention.

FIG. 2 is an applied view of the smart oximetry system of the present invention.

FIG. 3 is a flowchart of a smart oximetry method of the present invention.

FIG. 4 is a graph showing physical definitions of monitoring parameters according to the present invention during an incremental exercise.

FIG. 5 is a schematic drawing illustrating operation of the monitoring parameters entered into a neural network in one embodiment of the present invention.

### DETAILED DESCRIPTION OF THE INVENTION

Some embodiments of a smart oximetry method, a system thereof and a method for assessing a cardiovascular function based on a blood oxygen state with the technical features as described previously will be discussed below for illustrating the advantages of the present invention.

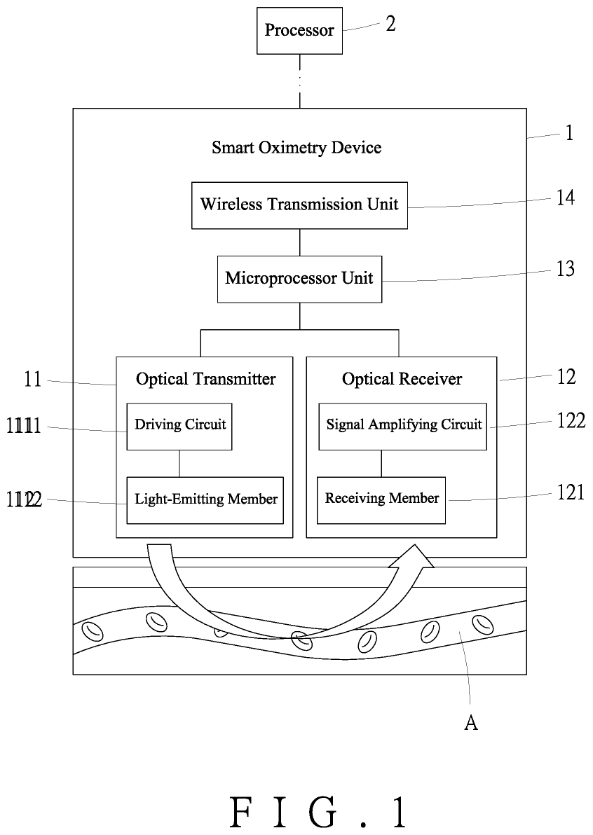

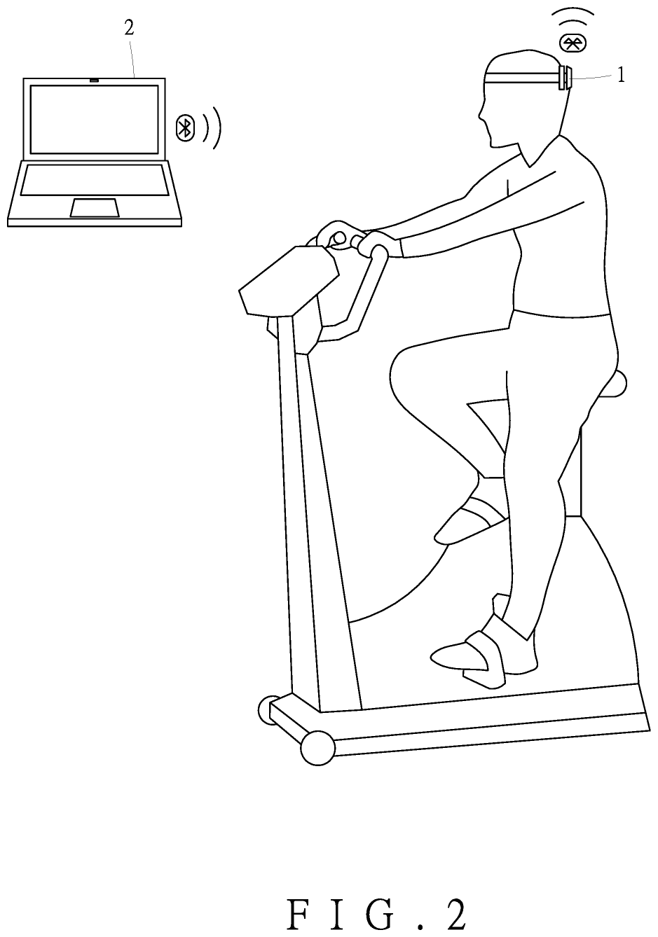

Referring to FIG. 1 through FIG. 3, a smart oximetry system of the present embodiment has a smart oximetry device (1) and a processor (2). The smart oximetry device (1) comprises the following components.

An optical transmitter (11) has a driving circuit (111) and a light-emitting member (112). The light-emitting member (112) emits a first light wave having a wavelength between 600 nm and 800 nm and a second light wave having a wavelength between 800 nm and 950 nm to a tissue (A) of a subject, which is the subject's brain tissue herein. An optical receiver (12) has a receiving member (121) and a signal amplifying circuit (122). The receiving member (121) receives a first reflected light wave and a second reflected light wave of the first light wave and the second light wave, respectively. A microprocessor unit (13) is electrically connected to the optical transmitter (11) and the optical receiver (12). The microprocessor unit (13) controls the optical transmitter (11) to emit the first light wave and the second light wave, and to receive the first reflected light wave and the second reflected light wave, so as to obtain a hemoglobin parameter. The hemoglobin parameter includes an oxy-hemoglobin concentration and a deoxy-hemoglobin concentration. A wireless transmission unit (14) is electrically connected to the microprocessor unit (13), for outputting the hemoglobin parameter to the processor (2).

The optical means used in the present embodiment for detecting the subject's blood oxygen concentration is one based on the modified Beer-Lambert law (MBLL). When light beams of different bands pass through solutions of different concentrations, light attenuation happens differently due to different absorbing and scattering properties of a substance and thus provides a basis for calculating the concentrations of the substance. Light attenuation is in linear relationship with the substance's molar extinction coefficient, the substance's concentration, and the optical path. Since MBLL is known in the art, the description about related calculation is omitted herein. The present embodiment uses the absorption differences of different absorptive substances related to light beams having wavelengths before and after their isosbestic points to obtain different absorption values. Therein, the near infrared band is mainly between 700 and 1400 nm. Light in this band can go deeper in human bodies and thus is favorable to measurement for deeper tissues with effectively reduced light attenuation. The isosbestic point of oxy-hemoglobin and deoxy-hemoglobin in the near infrared band between 600 and 1000 nm is about 800 nm. Thus, giving the forgoing range, the present embodiment uses light beams having wavelengths of 700 nm and 910 nm as two light sources.

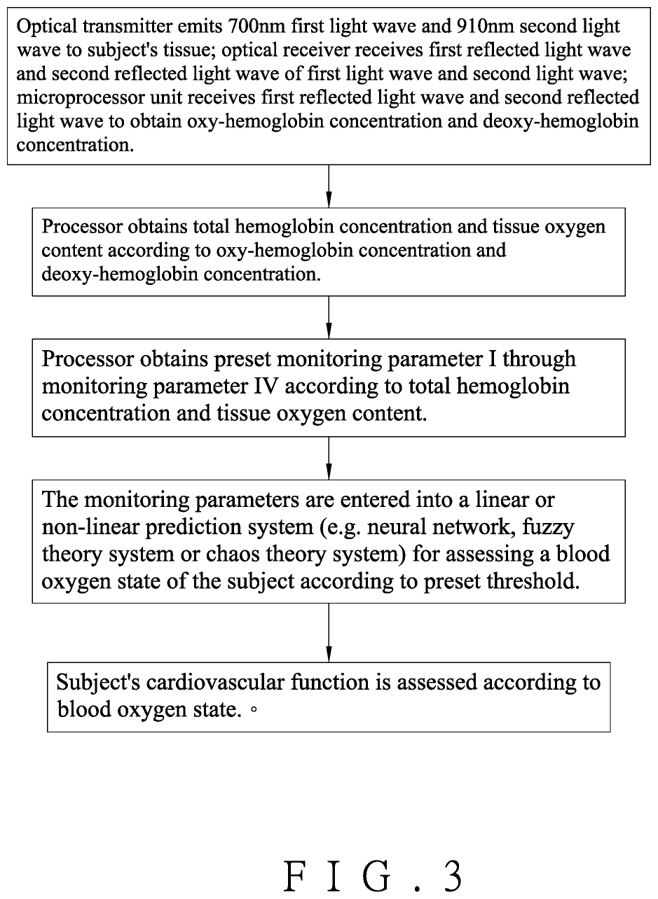

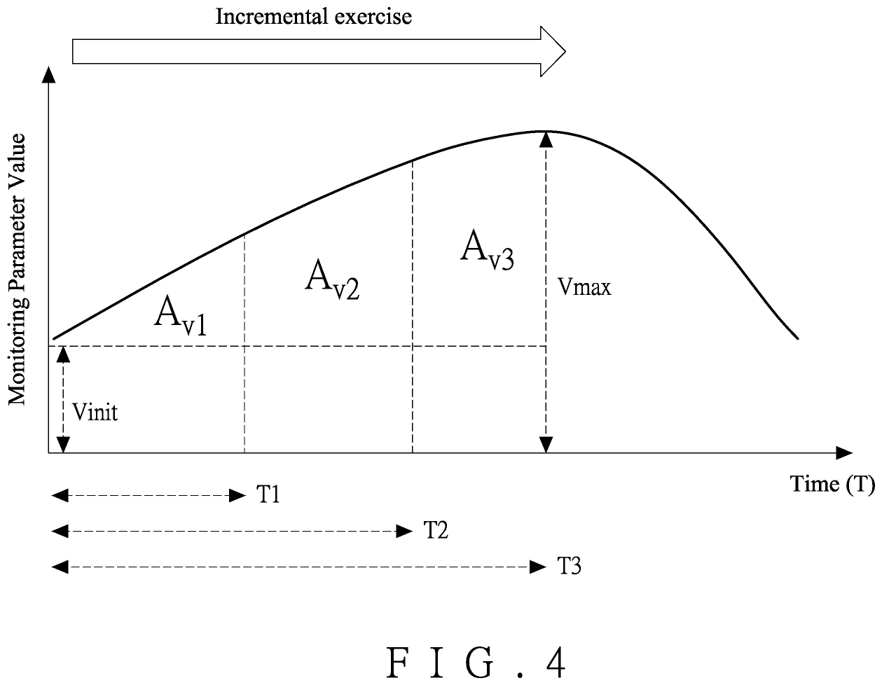

Referring to FIG. 1, FIG. 3 and FIG. 4, the processor (2) receives the oxy-hemoglobin concentration and the deoxy-hemoglobin concentration, and accordingly finds a total hemoglobin concentration and a tissue oxygen content, thereby and according to the total hemoglobin concentration and the tissue oxygen content calculates any one or a combination of the following monitoring parameters: a monitoring parameter I: a time period for the hemoglobin parameter to reach a peak value from an incremental exercise (T3); a monitoring parameter II: an amount of change of the hemoglobin parameter between an initial value and the peak value (Vmax-Vinit); a monitoring parameter III: an average rate of change of the hemoglobin parameter from the beginning of the incremental exercise to the peak value ((Aᵥ₁+Aᵥ₂+Aᵥ₃)/T3); and a monitoring parameter IV: an average rate of change of the hemoglobin parameter from a 60%-point of the entire incremental exercise to the peak value (Aᵥ₃/(T3-T2)).

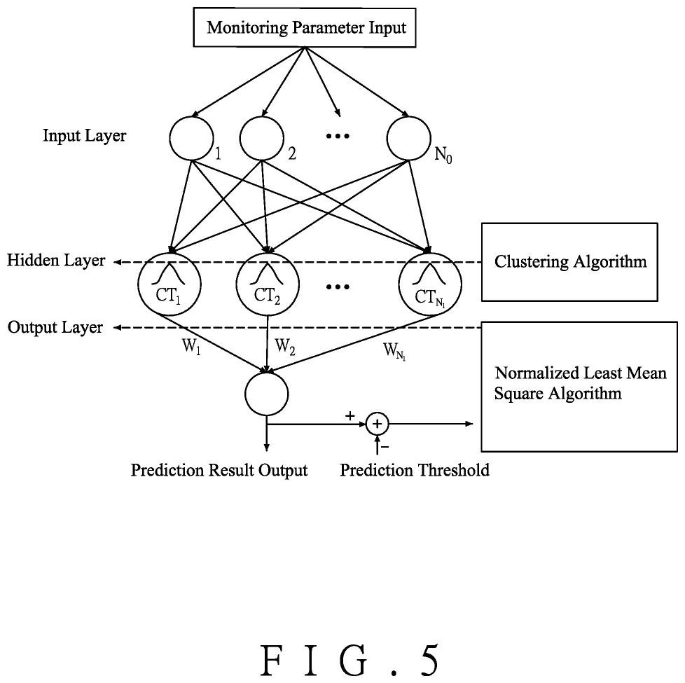

Referring to FIG. 1, FIG. 3 and FIG. 5, the monitoring parameters are into a linear prediction system or non-linear prediction system such as a neural network, a fuzzy theory system or a chaos theory system, and then assessment of a blood oxygen state of the subject is made according to a preset threshold. It is further possible to assess the subject's cardiovascular function according to the blood oxygen state, so that when any of the monitoring parameters exceeds the preset threshold, it is determined that the cardiovascular function of the subject is good, and otherwise, it is determined that the cardiovascular function of the subject is not good. In this way, medical staff can effectively divide cardiovascular function groups and recommend different exercise training and recovery processes for patients with cardiovascular diseases.

In an example where a radial basis function neural network is used, the radial basis function neural network has three structural layers, namely an input layer, a hidden layer, and an output layer. Therein, N₀ and N₁ are numbers of neurons in the input layer and the hidden layer. After trained using the k average clustering algorithm and the normalized least mean square algorithm, the values are compared with the preset threshold so as to determine the subject's blood oxygen state. Since operation in such a radial basis function neural network is known in the art, the related description is omitted herein for simplicity.

The present invention take a peak metabolic equivalent of 5 as a preset threshold for dividing patients with cardiovascular diseases into two groups, namely patients with relatively good cardiovascular function and patients with relatively poor cardiovascular function. The metabolic equivalent is for quantifying the energy consumed when a person is making a certain movement, and may be used to express an individual's aerobic exercise capacity. One metabolic equivalent is defined as the amount of oxygen uptake while sitting at rest and is about 3.5 ml/kg/min after weight normalization. Therein, oxygen uptake is related to cardiac arterial and mixed venous oxygen outputs. When the exercise reaches the maximal amount, the maximum oxygen uptake is the most important parameter for cardiopulmonary exercise testing, because it is recognized as the indicator of the limit of one's cardiorespiratory system for it reflects the subject's limitation in oxygen absorption, transportation and utilization. A plateau of oxygen uptake can be seen when a healthy person reaches his/her maximal exercise amount, which indicates the fact that when the maximal exercise amount is reached, maximum oxygen uptake can appear continuously. However, for patients unable to do vigorous exercise, there may be absence of a plateau of oxygen uptake during tests. Therefore, the peak oxygen uptake is usually used as a basis for estimating the maximum oxygen uptake. As revealed by some studies, tests of the maximal exercise amount may be considered as a useful method for risk stratification. Therein, patients with acute myocardial infarction are classified into the high-risk group. These patients usually have a peak metabolic equivalent of smaller than 5. Accordingly, assessment of a subject's cardiovascular function can be made.

In practical use of the present invention, according to experiments, the monitoring parameters were input to a radial basis function neural network for operation, and the results showed high classification accuracy in the subjects' cardiovascular function, which is as high as 90%.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A smart oximetry method, comprising:
acquiring a hemoglobin parameter related to a tissue of a subject;
calculating any one or a combination of the following monitoring parameters according to the hemoglobin parameter:
a monitoring parameter I: a time period for the hemoglobin parameter to reach a peak value from beginning of an incremental exercise;
a monitoring parameter II: an amount of change of the hemoglobin parameter between an initial value and the peak value;
a monitoring parameter III: an average rate of change of the hemoglobin parameter from the beginning of the incremental exercise to the peak value; and
monitoring parameter IV: an average rate of change of the hemoglobin parameter from a 60%-point of the entire incremental exercise to the peak value; and
entering the monitoring parameters into a linear prediction system or a non-linear prediction system, and assessing a blood oxygen state of the subject according to a preset threshold.

2. The smart oximetry method of claim 1, wherein the hemoglobin parameter comprises any one or a combination of a hemoglobin concentration and a tissue oxygen content.

3. The smart oximetry method of claim 2, wherein the hemoglobin concentration comprises an oxy-hemoglobin concentration, a deoxy-hemoglobin concentration and a total hemoglobin concentration, and the method further comprises beaming a first light wave having a wavelength between 600 nm and 800 nm and a second light wave having a wavelength between 800 nm and 950 nm to the tissue of the subject, receiving a first reflected light wave and a second reflected light wave of the first light wave and the second light wave, finding an oxy-hemoglobin concentration and a deoxy-hemoglobin concentration of the tissue according to the first reflected light wave and the second reflected light wave, and accordingly finding the total hemoglobin concentration and the tissue oxygen content.

4. The smart oximetry method of claim 3, wherein the monitoring parameters are calculated according to the total hemoglobin concentration and the tissue oxygen content.

5. The smart oximetry method of claim 1, wherein the tissue of the subject is a brain tissue of the subject.

6. The smart oximetry method of claim 1, wherein the linear prediction system or non-linear prediction system uses one of a neural network, a fuzzy theory system and a chaos theory system.

7. A smart oximetry system, for detecting a hemoglobin parameter of a tissue in a subject, so as to obtain a blood oxygen state of the subject, comprising:
a smart oximetry device, comprising:
an optical transmitter, for emitting a first light wave having a wavelength between 600 nm and 800 nm and a second light wave having a wavelength between 800 nm and 950 nm to the tissue of the subject;
an optical receiver, for receiving a first reflected light wave and a second reflected light wave of the first light wave and the second light wave;
a microprocessor unit, electrically connected to the optical transmitter and the optical receiver, the microprocessor unit controlling the optical transmitter to emit the first light wave and the second light wave, and to receive the first reflected light wave and the second reflected light wave, so as to obtain the hemoglobin parameter, the hemoglobin parameter including an oxy-hemoglobin concentration and a deoxy-hemoglobin concentration; and
a processor, signally connected to the smart oximetry device, the processor receiving the hemoglobin parameter, and calculating any one or a combination of the following monitoring parameters according to the hemoglobin parameter:
a monitoring parameter I: a time period for the hemoglobin parameter to reach a peak value from beginning of an incremental exercise;
a monitoring parameter II: an amount of change of the hemoglobin parameter between an initial value and the peak value;
a monitoring parameter III: an average rate of change of the hemoglobin parameter from the beginning of the incremental exercise to the peak value; and
a monitoring parameter IV: an average rate of change of the hemoglobin parameter from a 60%-point of the entire incremental exercise to the peak value;
wherein the monitoring parameters are entered into a linear prediction system or a non-linear prediction system for assessing a blood oxygen state of the subject according to a preset threshold.

8. The smart oximetry system of claim 7, wherein the hemoglobin parameter comprises a total hemoglobin concentration and a tissue oxygen content obtained from the oxy-hemoglobin concentration and the deoxy-hemoglobin concentration, and the processor calculates the monitoring parameters according to the total hemoglobin concentration and the tissue oxygen content.

9. A method for assessing a cardiovascular function based on a blood oxygen state, comprising:
acquiring a hemoglobin parameter related to a tissue of a subject, the hemoglobin parameter comprising a hemoglobin concentration and a tissue oxygen content;
calculating any one or a combination of the following monitoring parameters according to the hemoglobin parameter:
a monitoring parameter I: a time period for the hemoglobin parameter to reach a peak value from beginning of an incremental exercise;
a monitoring parameter II: an amount of change of the hemoglobin parameter between an initial value and the peak value;
a monitoring parameter III: an average rate of change of the hemoglobin parameter from the beginning of the incremental exercise to the peak value; and
a monitoring parameter IV: an average rate of change of the hemoglobin parameter from a 60%-point of the entire incremental exercise to the peak value; and
entering the monitoring parameters into a linear prediction system or a non-linear prediction system, and assessing a blood oxygen state of the subject according to a preset threshold, so that when any of the monitoring parameters exceeds the preset threshold, it is determined that the cardiovascular function of the subject is good, and otherwise, it is determined that the cardiovascular function of the subject is not good.
